(19) United States
(12) Patent Application Publication
(10) Pub. No.: US 2008/0300410 A1
(43) Pub. Date: Dec. 4, 2008

(54) Organic Compounds

(76) Inventors: Mahavir Prashad, Montville, NJ (US); Yugang Liu, Bridgewater, NJ (US); Bin Hu, Green Brook, NJ (US); Michael J. Girgis, Montville, NJ (US); Frank Schaefer, Rheinfelden (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/093,825

(22) PCT Filed: Nov. 16, 2006
(86) PCT No.: PCT/US06/60977

§ 371 Date: May 15, 2008

Related U.S. Application Data
(60) Provisional application No. 60/737,632, filed on Nov. 17, 2005.

(51) Int. Cl. C07D 211/32

(52) U.S. Cl. 546/234

(57) ABSTRACT

The present invention relates to salts of aryl compounds as discussed below and to methods of manufacture thereof, as well as other subject matter. More particularly, the invention relates to salts useful as intermediates for the synthesis of the cinnamanilide of formula (Y): where R^{a} is selected from H, OH, C₁, C₂, C₃ or C₄ alkyl; and R¹ is C₁, C₂, C₃ or C₄ alkyl.

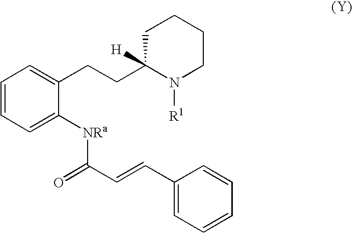

### BACKGROUND

The present invention relates to salts of aryl compounds as discussed below and to methods of manufacture thereof, as well as other subject matter.

More particularly, the invention relates to salts useful as intermediates for the synthesis of the cinnamanilide of formula (Y):

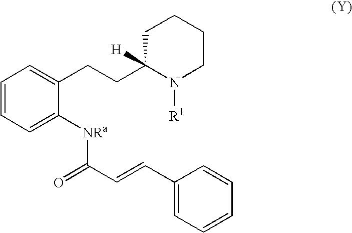

where R^{a} is selected from H, OH, C₁, C₂, C₃ or C₄ alkyl; and R¹ is C₁, C₂, C₃ or C₄ alkyl.

The compounds of formula (Y) are well described in the art. Examples of syntheses of compounds of formula (Y) are described in EP 0973741 and U.S. Pat. No. 3,931,195, which are incorporated herein by reference.

The compounds of formula (Y) may be used as a 5-HT₂ antagonist, for example. In particular, compound (Y1) below may be mentioned. Furthermore, compound (Y1) may be used as a pharmaceutical agent for treating 5-HT₂-related diseases such as haemorrhoids, for example.

Accordingly, the salts of the present invention may act as intermediates in processes for the manufacture of compounds of formula (Y).

In particular the invention further relates to intermediates for the synthesis of 2′[2-1-(methyl -2-piperidyl) ethyl] cinnamanilide (Y1), which is a compound of formula (Y) where R^{a} is hydrogen and R¹ is methyl:

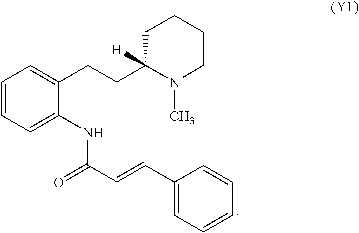

Processes for the preparation of compounds of formula (Y) are described in U.S. Pat. No. 3,931,195 which comprises the step of alkylating compounds of formula (i) (below) with an alkyl halide, such as methyl iodide for methylation. The same methylation step is described in EP0973741 for the synthesis of compound (Y1).

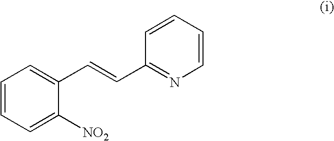

Thus, the processes described in the prior art involve the use of a highly toxic reagent (e.g. methyl iodide) and provides a yield of about 50%.

### SUMMARY OF THE PRESENT INVENTION

The present invention provides an alternative synthesis to the prior art which overcomes the environmental and health implications of using an alkyl halide together with a surprising increase in yield. The process of the present invention provides a new route to compounds of formula (Y) via a new intermediate salt.

Accordingly, the present invention relates to salts of Formula (I) and to methods of manufacture thereof:

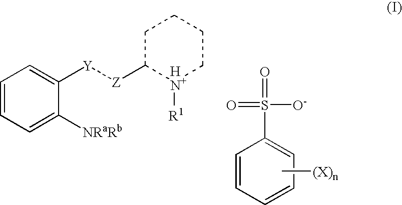

where X is an organic or inorganic moiety,
n is 0, 1, 2, 3 or 4; and
R^{a} and R^{b} are each independently selected from H, OH, C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl, or are both oxygen to produce the moiety NO₂; and
R¹ is C₁, C₂, C₃ or C₄ alkyl; and
Y and Z are both carbon; and
the broken lines [symbol] represent saturated or unsaturated bonds.

Salts of formula (I) may be intermediates in the process of forming cinnamanilide (Y).

Thus, the salts of the present invention may be precursors to a pharmaceutical composition containing cinnamanilide (Y). It will be understood that, in pharmaceutical compositions containing cinnamanilide (Y), the cinnamanilide may be in the form of a pharmaceutically acceptable salt or prodrug thereof and that, accordingly, the compounds of formula (1) may be used as intermediates in forming such salts of prodrugs.

Therefore, pharmaceutical products, for example combinations and/or compositions, containing a cinnamanilide (Y), which has been synthesised via an intermediate of formula (I), may contain trace amounts of salts of formula (I) (less than or equal to 1000 ppm, 100 ppm or 10 ppm, for example) as contaminants.

In another aspect, the invention relates to the manufacture of salts of formula (I), as illustrated below in Scheme 1:

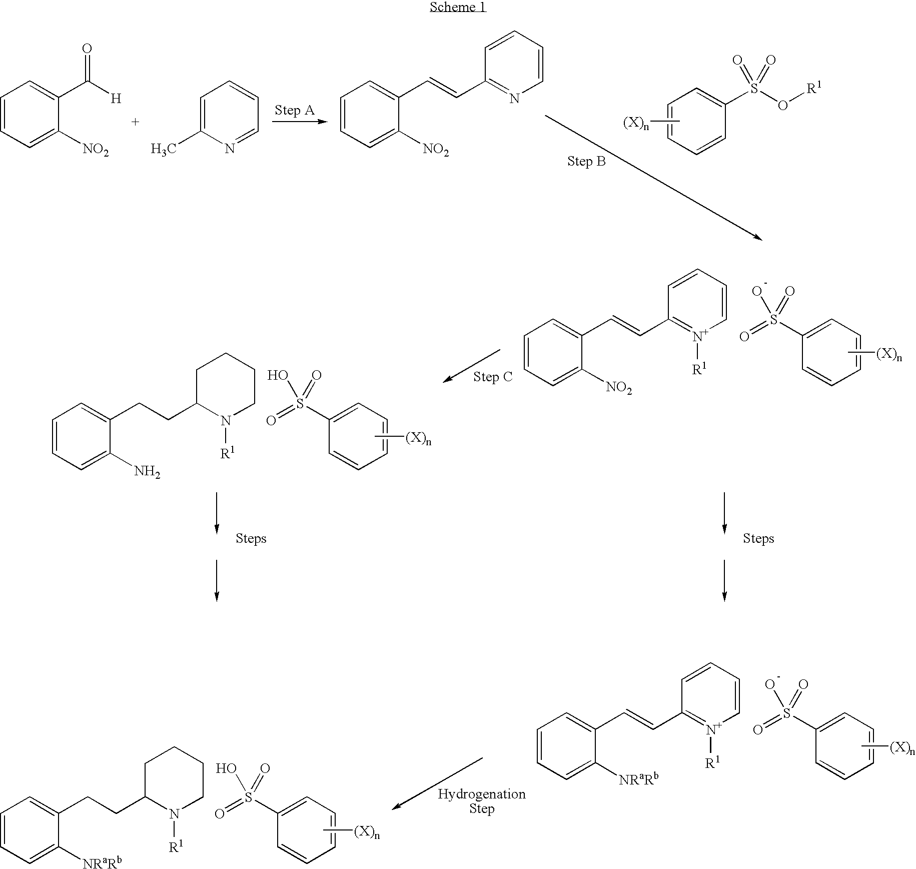

where R¹, X and n are as herein defined.

An exemplary scheme, where the final product is a compound of formula (Y1), is shown below:

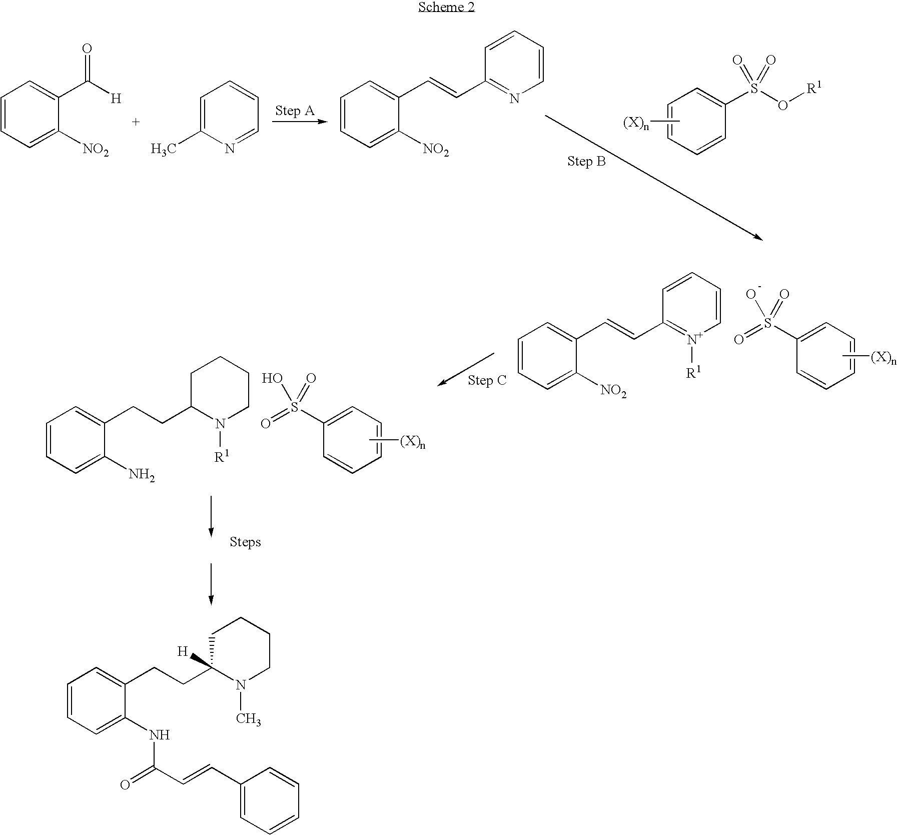

wherein R¹, X and n are as herein defined.

### DETAILED DESCRIPTION OF THE INVENTION

### The Salts

The present invention relates to salts of formula (I):

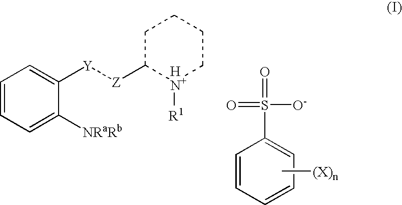

where X is an organic or inorganic moiety,
n is 0, 1, 2, 3 or 4; and
R^{a} and R^{b} are each independently selected from H, OH, C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl, or are both oxygen to produce the moiety NO₂; and
R¹ is C₁, C₂, C₃ or C₄ alkyl; and
Y and Z are both carbon; and
the broken lines [symbol] represent saturated or unsaturated bonds.

Particular examples of compounds falling with in the scope of formula (I) are shown below as formulae (IA) and (IIB):

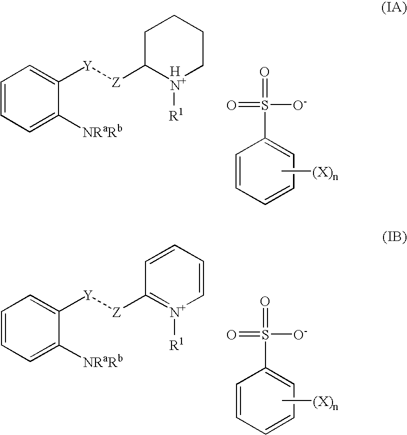

In one aspect of the present invention, the salts have the formula (II):

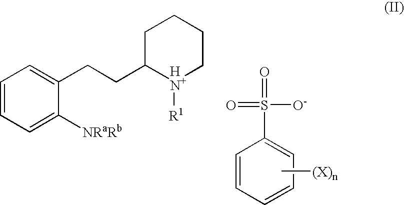

where X, n, R^{a} and R^{b} and R¹ are as defined above in Formula (I). R^{a} and R^{b} are normally not oxygen, e.g. are independently H or alky).

In a second aspect of the present invention, the salts have a formula of (III):

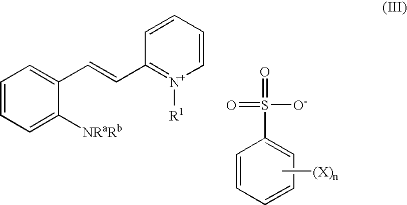

where X, n, R^{a} and R^{b} and R¹ are as defined above in Formula (I). R^{a} and R^{b} are normally both oxygen.

The present invention does not exclude salts having the formula (IV):

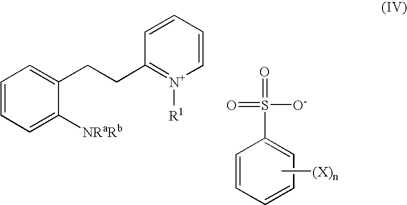

where X, n, R^{a} and R^{b} and R¹ are as defined above in Formula (I). Compounds of formula (IV) may be by-products of the conversion of salts of formula (III) to salts of formula (II). It is contemplated that the salts of formula (II) may contain trace amounts of salts of formula (IV).

In one class of salts, X is selected from —OH, NR^{c}R^{d}, halogen, C₁, C₂, C₃ or C₄ alkyl, C₁, C₂, C₃ or C₄ haloalkyl, C₁, C₂, C₃ or C⁴ alkoxy, C₁, C₂, C₃ or C₄ alkenyl.

R^{c} and R^{d} are each independently selected from hydrogen, —OH, C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl.

Halogen may be selected from chloro, fluoro, bromo and iodo, e.g. chloro or fluoro.

The organic moiety, for example C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl, may be substituted or unsubtituted.

In a further class of salts, n is 1.

A preferred substituent X is alkyl. In particular, X is methyl.

R^{a} and R^{b} may be protecting groups. The protection of functional groups by such protecting groups, suitable reagents for their introduction, suitable protecting groups and reactions for their removal will be familiar to the person skilled in the art. Examples of suitable protecting groups can be found in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4th edition, Vol. 15/I, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974.

In one class of compounds R^{a} and R^{b} are oxygen. In another class of compounds R^{a} and R^{b} are hydrogen.

R¹ is preferably methyl.

The ring represented by broken lines [symbol] is preferably one of piperidine and pyridine.

In one embodiment of the present invention, X is methyl and the salts of the present invention have the formula (Ia):

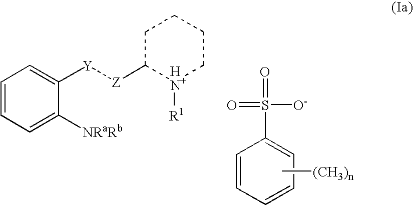

where R¹ n, R^{a} and R^{b} are as hereinbefore described, e.g. in formula (I)

In exemplary embodiments, the salts of the present invention have the formulae:

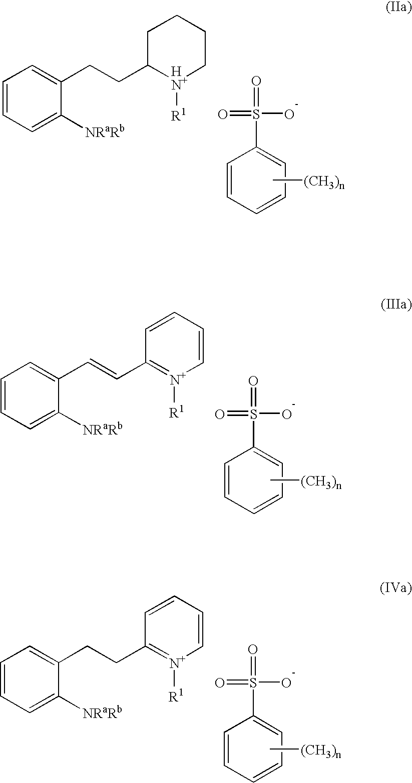

where R¹ n, R^{a} and R^{b} are as hereinbefore described, e.g. in formula (I). In formula (IIa), R^{a} and R^{b} are normally both not oxygen, e.g. independently selected from H and alkyl. In formula (IIIa), R^{a} and R^{b} will normally be oxygen.

In a second embodiment of the present invention, there are provided salts of formula (Ib), where both X and R¹ are methyl:

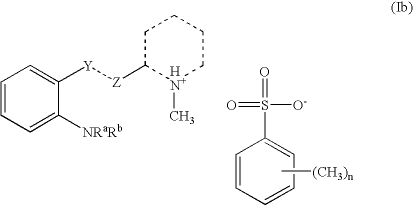

In exemplary embodiments, the salts of the present invention have the formulae:

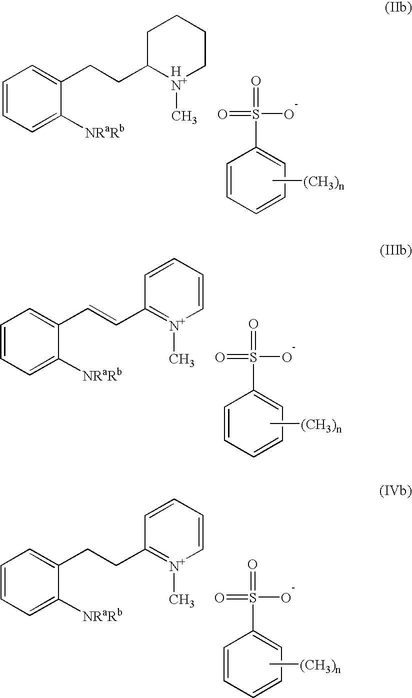

where R¹ n, R^{a} and R^{b} are as hereinbefore described, e.g. in formula (I). In formula (IIb), R^{a} and R^{b} are normally both not oxygen, e.g. independently selected from H and alkyl. In formula (IIIb), R^{a} and R^{b} will normally be oxygen.

In a particularly preferred embodiment, the benzene sulphonate comprises an X substituent which is meta or para to the SO₃ group. Particularly preferred is para. In some salts, there is exactly one X moiety and it is in the para-position.

In a third embodiment, the salts of the present invention have a formula (Ic):

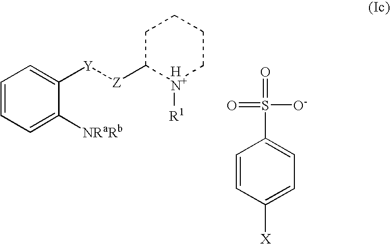

In exemplary embodiments, the salts of the present invention have the formulae:

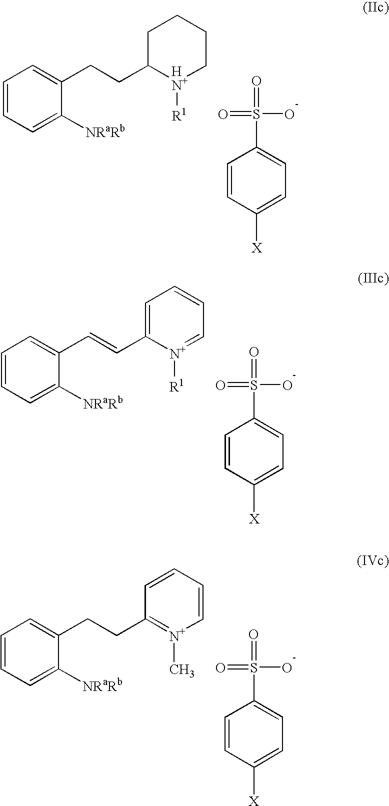

where R¹ n, R^{a} and R^{b} are as hereinbefore described, e.g. in formula (I). In formula (IIc), R^{a} and R^{b} are normally both not oxygen, e.g. independently selected from H and alkyl. In formula (IIIc), R^{a} and R^{b} will normally be oxygen.

In a fourth embodiment, the salts of the present invention have a formula (Id):

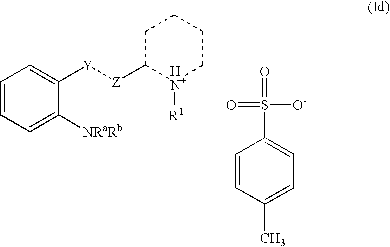

In exemplary embodiments, the salts of the present invention have the formulae:

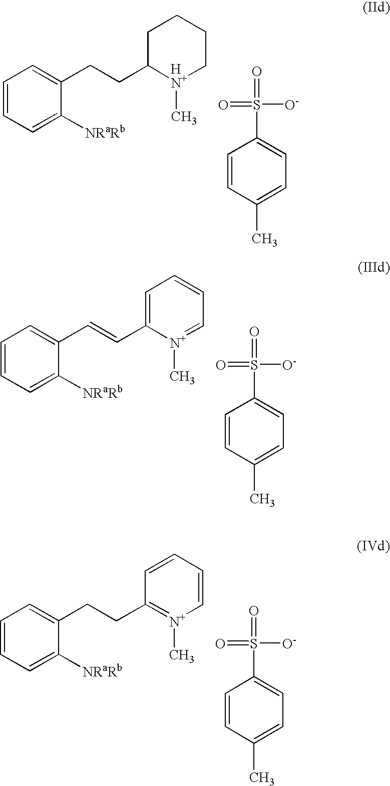

where R¹ n, R^{a} and R^{b} are as hereinbefore described, e.g. in formula (I). In formula (IId), R^{a} and R^{b} are normally both not oxygen, e.g. independently selected from H and alkyl. In formula (IIId), R^{a} and R^{b} will normally be oxygen.

As mentioned above, for salts of formulae (IIa-d), R^{a} and R^{b} are preferably hydrogen, thus NR^{a}R^{b} is NH₂ and or salts of formulae (IIIa-d), R^{a} and R^{b} are preferably oxygen and thus NR^{a}R^{b} is NO₂.

Where a salt of the present invention is a salt of formulae IIId or IId, the salt is useful as an intermediate in a process for forming compound (Y). It is contemplated that the compounds of formula (IVd) may also function as intermediates.

In one aspect, R^{a} and R^{b} are H and the salt IId is useful as an intermediate in a process for forming the compound (Y1).

In another aspect, R^{a} and R^{b} are oxygen and the salt IIId is useful as an intermediate in a process for forming the compound (Y1).

Particular compounds of the present invention may be represented by formulae (IIe) and (IIIe) below:

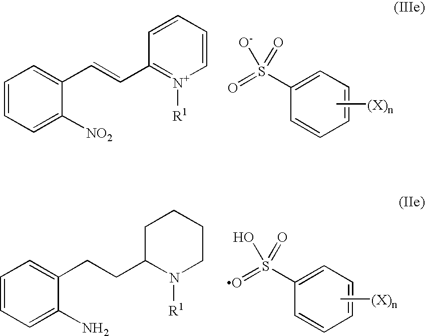

Examples of specific compounds of formula II and III are shown below as formulae (IIf) and (IIIf) below:

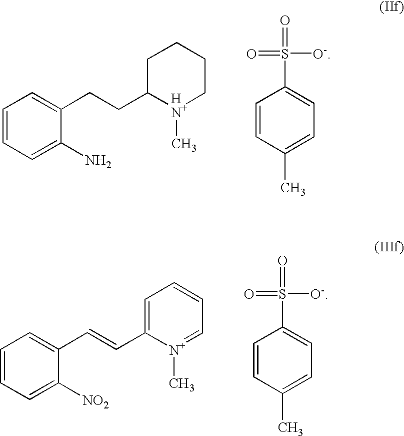

In one process, a salt (II) may be reacted with cinnamoyl chloride to form a salt of the formula (Y).

Preferably, salts of formula (III) are precursors to salts of formula (II), where the salts of formula (III) are hydrogenated, for example.

When salts of Formula (III) have been hydrogenated typically, the bond Y-Z is saturated and R^{a} and R^{b} are hydrogen. Typically, NR^{a}R^{b} is converted from NO₂ to NH₂ during a hydrogenation process. The ring represented by broken lines [symbol] is also typically saturated after hydrogenation.

The present invention also relates to a product, for example a solution, comprising a source of cations of formula (vi) and a source of anions of formula (vii):

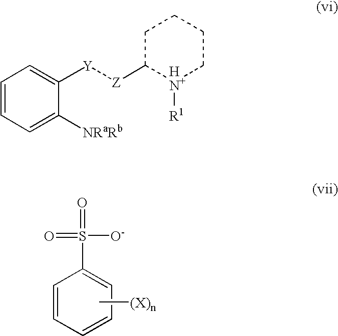

where X is an organic or inorganic moiety,
n is 0, 1, 2, 3 or 4; and
R^{a} and R^{b} are each independently selected from H, OH, C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl, or are both oxygen to produce the moiety NO₂; and
R¹ is C₁, C₂, C₃ or C₄ alkyl; and
Y and Z are both carbon; and
the broken lines [symbol] represent saturated or unsaturated bonds.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, salts, chemical moieties or groups described in conjunction with a particular aspect, embodiment or example of the invention are to be understood to be applicable to any other aspect, embodiment or example described herein unless incompatible therewith.

The disclosure includes prodrugs for the active pharmaceutical species of the disclosure, for example in which one or more functional groups are protected or derivatised but can be converted in vivo to the functional group, as in the case of esters of carboxylic acids convertible in vivo to the free acid (which representation includes tetrahedrol boronate species, as discussed below), or in the case of protected nitrogens. The term “prodrug,” as used herein, represents in particular compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996), each of which is incorporated herein by reference.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amine | Amides, carbamates, imines, enamines, |
| Boronic acid | Diol ester |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:
Oxidative activation
   N— and O— dealkylation
   Oxidative deamination
   N-oxidation
   Epoxidation
Reductive activation
   Azo reduction
   Sulfoxide reduction
   Disulfide reduction
   Bioreductive alkylation
   Nitro reduction.

Also to be mentioned as metabolic activations of prodrugs are nucleotide activation, phosphorylation activation and decarboxylation activation.

### The Process

The present invention relates to a process for manufacturing a salt of the present invention, where the process may comprise
(a) reacting 2-nitrobenzaldehyde with 2-picoline to form a salt of formula (i);

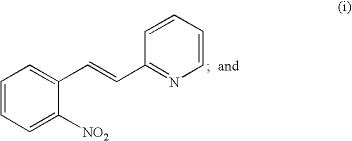

(b) converting the salt of formula (i) into a salt of the present invention, for example any of salts of the formulae IIa-f or IIIa-f.

The salt of formula (i) may be treated with a base to raise the pH to 9 or more, for example between 9 and 11, prior to converting it to a salt of the present invention.

The salt of formula (i) may be isolated during the process of the present invention.

In another aspect of the present invention, the salt of formula (i) may be treated with an alkylating agent as part of the process of converting it to a salt of the present invention, as described herein. The alkylating agent may be, for example, a substituted benzyl sulphonate of formula (iv):

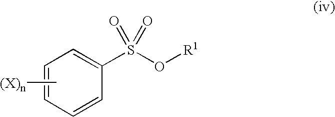

where R¹ is C₁, C₂, C₃ or C₄ alkyl and X is an organic or inorganic moiety and n is 1, 2, 3 or 4.

In a further aspect of the present invention, there is provided a process for converting any unsaturated bonds represented by the broken lines [symbol] in a salt of the present invention, e.g. an unsaturated salt of the present invention, of which salts of formulae (III) may be mentioned, to a saturated salt of the present invention, by exposing the unsaturated salt to hydrogenation conditions comprising a pressure of, for example, over about 5 bar and/or a temperature not above about 40° C., for example.

The resulting product from the hydrogenation process may be any one of salts IIa-d or IVa-f. In particular, the salts IIa-f may be mentioned.

The processes of the present invention may be suitably scaled up to an industrial scale.

The reaction of the present invention may, for example, be conducted in an inert atmosphere, for example under a nitrogen atmosphere.

Where an increase in temperature is described, it is contemplated, unless otherwise stated, that such an increase may for example be conducted at a level of 0.5-2° C. per minute, for example 1° C. per minute.

As mentioned hereinbefore, the salts of the present invention may be synthesised by the process as shown in Scheme 1. This process is now described in more detail below. Where illustrated, the symbols R¹, X and n are as hereinbefore described, i.e. R¹ is C₁, C₂, C₃ or C₄ alkyl and X is an organic or inorganic moiety and n is 1, 2, 3 or 4.

### Step A

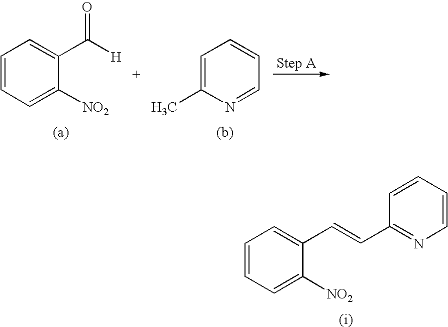

A mixture, e.g. solution, of 2-nitrobenzaldehyde (a) and 2-picoline (b) in dehydrating conditions, for example, in the presence of a carboxylic acid anhydride, such as acetic anhydride, for example, is allowed to react, typically stirred, for example under inert, e.g. nitrogen conditions. The reaction is suitably performed at an elevated temperature. The mixture may then therefore be heated to an internal temperature of 130 to 145° C., for example 135 to 140° C., typically 138° C. (jacket temperature of approximately 135 to 150° C.). The mixture may be heated over a period of, for example, 40 minutes up to a period of about 30 hours, for example. The reaction may be stirred. The reaction may be monitored by HPLC for the disappearance of 2-nitrobenzaldehyde. The reaction is assumed complete when ≦4% (peak area) of 2-nitrobenzaldehyde remains unreacted. The reaction mixture may be stirred at the aforementioned internal temperature (typically, 138±2° C.) for an additional three hours until the peak area limit (≦4%) is reached. The heated reaction mixture may be stirred. The heated reaction mixture may undergo reflux. Preferably, the heated reaction mixture undergoes gentle reflux. After the reaction is complete the dark reaction mixture may be cooled to an internal temperature of 0 to 20° C., for example 5-15° C., typically 10° C. over a period of one hour. Then, water is added over a period of at least about 20 minutes, for example, while maintaining an internal temperature of 0 to 40° C., for example 5 to 35° C. The reaction mixture may then be cooled to an internal temperature of approximately 15° C. over a period of 10 minutes, for example, and a base, e,g, sodium hydroxide, (for example, 50% v/v) is added over a period of 40 minutes, for example, whilst maintaining an internal temperature of 10 to 40° C., for example 15 to 35° C. (typically a jacket temperature of 35° C.). Typically the pH of the solution is 7 or more, for example 8 or more, typically 9 to 11. The resulting reaction mixture may then be seeded with 2-[(E)-2-(2-nitrophenyl)ethenyl]-pyridine. Then, a base, e.g. sodium hydroxide, (for example 50% v/v) may be added over a period of about 20 minutes, for example, whilst maintaining an internal temperature of 10 to 40° C., for example 15 to 35° C. The pH of the reaction mixture may be checked after addition of approximately 90% of the total amount of sodium hydroxide solution has been added. The final pH is usually 7 or more, for example 8 or more, typically 9 or more, for example from 9 to 11. The reaction mixture may then be stirred at this temperature for an additional one hour. The resulting solid may be collected e.g. by filtration with suction. The collected solid may then be washed with water (for example four times). The solid may then be dried under reduced pressure (15 to 40 mbar) at 60 to 80° C., typically at 70° C. for approximately 16 hours. Typically, the solid is dried until an LOD of less than 1% is reached.

In this step, the product is crystallised directly from the reaction mixture. Thus this reaction step avoids the need for a separate recrystallisation step, if required. The purity of the product may not be affected by the absence of a recrystallisation step. The product purity is preferably over 90%, for example over 90%, typically over 95%, such as 98%, for example.

A by-product, acetic acid, formed from the acetic anhydride present may be removed by the addition of a base, for example sodium hydroxide. Thus, basifying the reaction mixture in the final step serves to remove any acid by-product present. It is therefore contemplated that the product of this reaction contains only trace (less than 100 ppm) amounts of carboxylic acid, typically acetic acid.

### Step B

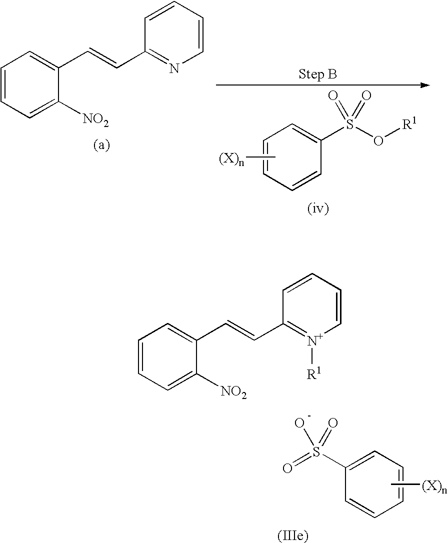

A mixture, e.g. solution, of 2-[(E)-2-(2-nitrophenyl)ethenyl]-pyridine (a) in a solvent, e.g. acetonitrile, together with an alkylating agent, for example an aryl sulphonate (iv) e.g. a toluene sulphonate, typically methyl p-toluenesulfonate, is allowed to react, e.g. may be stirred, for example in an inert atmosphere. Then, further solvent, typically acetonitrile, may be added. The reaction is suitably performed at an elevated temperature. The mixture may then therefore be heated to an internal temperature of 75 to 90° C., for example 80 to 88° C., typically 83° C. (jacket temperature 90 to 95° C., for example). At this temperature, the reaction mixture may undergo a gentle reflux. The reaction mixture may be heated for a period of 30 to 60 minutes, for example 35 to 45 minutes, typically 40 minutes and the reaction mixture may be stirred at this temperature for an additional 24 hours. The reaction mixture may then be cooled to an internal temperature of 30 to 50° C., for example 35 to 45° C. (jacket temperature 35 to 45° C., for example) over a period of 30 minutes, for example. The reaction may be monitored by HPLC. The reaction may be assumed to be complete when the peak area is less than 3% by HPLC of the pyridine reactant remains. The reaction mixture may be stirred at an internal temperature of 84±3° C. for an additional 2 hours until this limit is reached. Then, the reaction mixture may be heated to an internal temperature of 75 to 85° C., for example 80±3° C. (jacket temperature 80 to 83° C., for example). The mixture may be heated over a period of 20 minutes, for example. Then, an amount of an alkyl acetate, typically isopropyl acetate may be added over a period of 20 to 40 minutes, typically 30 minutes, whilst maintaining the internal temperature at 65 to 85° C., for example 70 to 83° C. (jacket temperature 80 to 83° C., for example). Solids should precipitate out when the, isopropyl acetate, for example, is added. Crystallisation may be induced by stirring at this point and the addition of isopropyl acetate halted. Once stirring has been commenced, the rest of the isopropyl acetate may be added. The resulting reaction mixture, for example suspension, may then be cooled to an internal temperature of 10 to 30° C., for example 15 to 25° C., typically 20° C. The reaction mixture may be cooled over a period of 1 hour. Then, the cooled, e.g. suspension, may be stirred at this temperature for an additional 4 hours, for example. The resulting solid may then be collected by filtration. The solid may be washed by isopropyl acetate (for example twice). The product (IIIe) may be dried under reduced pressure (15 to 40 mbar) at 60° C., for example. The product (IIIe) may be considered dry once the LOD is below 1%.

Preferably, this process provides a much improved yield over the prior art. In particular, the present process provides a much increased yield compared with the prior methodology of heating the product (iii) in acetone and methyl iodide, as has been described in the art (EP 0973741).

The present invention does not require the use of the toxic methyl iodide. The use of p-toluenesulphonate provides the desired product.

The present invention therefore provides an alternative process to the prior art, where highly toxic methyl iodide is replaced with an aryl sulphonate as an alkylating agent. It is surprising that the use of the aryl sulphonate is effective in producing a high yield and high purity products. In addition, the use of the aryl sulphonate in place of the alkyl halide of the prior art removes the environmental and health problems associated with such alkylating agents. It is therefore contemplated that the use of an aryl sulphonate as described herein may be scaled up to an industrial scale providing an economically viable and environmentally friendly solution to the existing problems experienced with the prior art.

Reactions using dimethyl sulphate in place of the aryl sulphonate (iv) in acetone afforded an analogue of product (IIIe) whose counterion was hydrogen sulfate, not methyl sulphate as would be expected. It is therefore asserted that the initially formed methyl sulfate counterion could react further with acetone to generate hydrogen sulfate. Changing the solvent to isopropyl acetate, the yield was significantly improved, affording an analogue of (IIIe) with methyl sulfate as the counterion. However, in the next step, step C, is a hydrogenation reaction in methanol at elevated temperature, the methyl sulfate salt of (IIIe) could potentially generate dimethyl ether and therefore, this may prove to indicate a further advantage of the aryl sulphonate (iv).

The use of alkyl p-toluenesulfonate as the alkylating agent is preferred. In particular, the use of methyl p-toluenesulfonate as a methylating agent is preferred.

The preferred solvent for reaction is acetonitrile, although isopropyl acetate or toluene are contemplated.

Any residual starting material (a) in the product could potentially cause purification problems in the next step. In acetonitrile, however, not only is the reaction homogenous but the conversion is about 98%.

Residual starting material (a) was efficiently removed during crystallization.

| | Conversion | Yield | Purity |
|---|---|---|---|
| Conditions (equiv) | (%) | (%) | (%) |
| (a) (1), Me₂SO₄ (2.2), | 84 | 61 | 98 |
| acetone, reflux | | | |
| (a) (1), Me₂SO₄ (1.5), | 95 | 83 | 98 |
| iPrOAc, reflux | | | |
| (a) (1), (iv) (1.5), acetone, | 90 | — | — |
| reflux | | | |
| (a) (1), (iv) (1.5), iPrOAc, | 95 | 85 | 97 |
| reflux | | | |
| (a) (1), (iv) (1.5), Toluene, | 97 | >90 | 97 |
| reflux | | | |
| (a) (1), (iv) (1.5), iPrOH, reflux | ~45 | — | — |
| (a) (1), (iv) (1.5), acetonitrile, | 98 | 92 | >98 |
| 84° C. | | | |

In addition, the preferred methylating agent is an aryl sulphonate, e.g. p-toluenesulfonate, since, in the later reaction step C, where p-toluenesulfonate is the counterion, salt of formula (I) may be more easily isolated.

The product purity is preferably over 90%, for example over 90%, typically over 95%. Particularly preferably, the purity is >98% by HPLC.

Preferably, the product only has a trace of the solvents used in the process step. For example, the product preferably has less than 1000 ppm, 100 ppm or 10 ppm of acetonitrile and isopropyl acetate present, e.g. after drying. Moreover, in a particular class of salts, the final product has no detectable traces of methyl iodide present.

### Step C

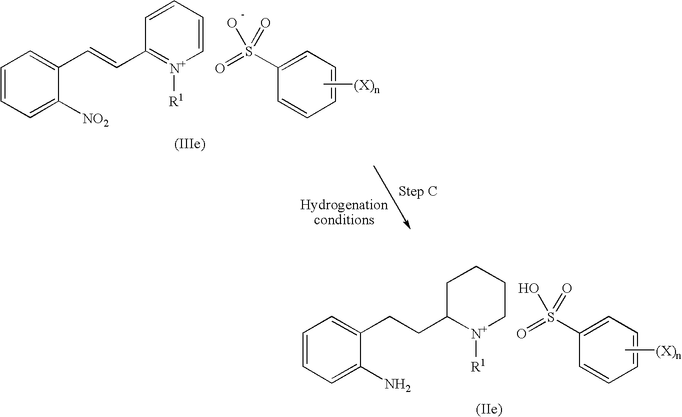

In general terms, this step is a hydrogenation step. A vessel has an inert atmosphere, achieved by, for example, pressurising with nitrogen to 4.5 bar, then depressurising to 1 bar and repeating this pressurisation/depressurisation four times. The product (IIIe) from step B may then be added to the vessel. After addition of the aforementioned product, the vessel may then be pressurised/depressurised a further four times with nitrogen. Then, a catalyst, e.g. Pd or Pt, preferably in the presence of carbon, for example 10% Pd/C is added to the vessel. The vessel may then once again be pressurised and depressurised four times with nitrogen. Then, an alcohol, for example methanol may be added. Then, the vessel may once again be pressurised/depressurised four times with nitrogen. Each pressurisation step may be up to 5 bar, for example up to 4.5 bar. The depressurisation may be down to 1 bar.

The vessel may then be stirred or otherwise agitated, at a rate sufficient to obtain at least partial suspension of the catalyst, e.g. full suspension of the catalyst, for example at a rate of about 450 rpm, and the temperature may be set at 25 to 35° C., for example 30° C. The temperature may be allowed to equilibrate at about 30° C. Stirring may then be stopped once equilibrium has been reached. The nitrogen may then be replaced with hydrogen by pressurising the vessel with hydrogen to 4.5 bar and then depressurising to 1 bar. The pressurisation/depressurisation cycle may be carried out a further four times. The agitator (or stirrer) may be turned off during hydrogen introduction to prevent hydrogen reaction from occurring at an early stage. After the final depressurisation, the vessel may be pressurised to about 3-5 bar, for example about 5 bar, typically 5.2 bar, by the introduction of nitrogen for example, and agitated at a rate sufficient to obtain at least partial suspension of the catalyst, e.g. full suspension of the catalyst, for example at a rate of about 450 rpm.

The agitation may serve to start the reaction. The initial reaction is exothermic, giving a maximum heat evolution rate of about 35 W/kg (except for a short-lived spike with a maximum of about 50 W/kg). The reaction may be detected by hydrogen uptake and heat evolution. The hydrogenation process may be carried out at about 30° C. and about 5.2 bar for about 5-10 hours, for example 7-8 hours, typically 7.2 hours. Then, the vessel may be depressurised to 1 bar and purged with nitrogen, by pressurising to 4.5 bar and depressurising as aforementioned. A total of five pressurisation/depressurisation cycles may be conducted. The reactor may then be emptied and rinsed with an alcohol, e.g. methanol. The alcohol rinse may then be combined with the reaction mixture. The final batch may then be filtered e.g. over a pad of celite or other filter. The filter, e.g. celite pad, may then be washed with further alcohol e.g. methanol and the filtrate combined. The filtrate may then be distilled at an internal temperature of 30 to 50° C., for example 35 to 45° C. (jacket temperature 65 to 75° C.) under reduced pressure (80 to 160 mbar) to a volume of about one third. To the reduced-volume filtrate may be added a peroxide-free alcohol, e.g. 2-propanol. The reaction mixture may then be distilled at an internal temperature of 30 to 50° C., e.g. 35 to 45° C. (jacket temperature 65 to 75° C.) under reduced pressure (80 to 160 mbar) to approximately one third. The reduced-volume mixture is then heated to an internal temperature of 40 to 80° C., e.g. 50 to 70° C., typically 60±5° C., for example, over a period of about 20 minutes and then an alkyl acetate, typically isopropyl acetate may be added, for example, over a period of about 20 minutes while maintaining the internal temperature at about 55 to 65° C., for example. The reaction mixture may then be cooled to an internal temperature of about 40±5° C., for example, over a period of about 20 minutes and the mixture seeded with a small amount of the product. The resulting mixture, e.g. suspension, may be cooled to an internal temperature of about 20±5° C., for example, over a period of about 1 hour and stirred at this temperature for an additional 4 hours for example. The resulting solid may then be collected by filtration and optionally washed with a solvent, for example a mixture of solvents, which may be a mixture of an alcohol and alkyl acetate, typically 2-propanol and isopropyl acetate. The solvent is preferably in a mixture of alcohol: acetate of 1:2 v/v. The solid is optionally washed two times. The solid may then be dried under reduced pressure (15 to 49 mbar) at approximately 60° C. The drying is completed once the LOD is less than 1%.

The preferred hydrogenation conditions include 10% Pt/C (65% wet) with 2.5% loading.

The hydrogenation reaction of step C is carried out at a high pressure, which may provide a route for higher selectivity for the desired product.

The reaction temperature is maintained at a relatively low level in order to favour the formation of the desired product. It was found by the present inventors that increasing temperature increased by-products, in particular a products such as A and B, below.

The reaction is preferably agitated at a rate of between 100 and 300 rpm, for example 150 to 250 rpm, typically 170 to 200 rpm. The rate of agitation may be directly related to mass transfer.

This process step provides the option of using a process at a constant temperature and pressure. By providing constant temperature and pressure, the present process step allow the reduction of the production undesired side reactions such as one between the unconverted reactant and the initial nitroso intermediate, for example. Such side reactions can produce by-products such as products A and B below. The present invention therefore provides a route to reduce, preferably minimise these side reactions and provide high purity products as disclosed herein:

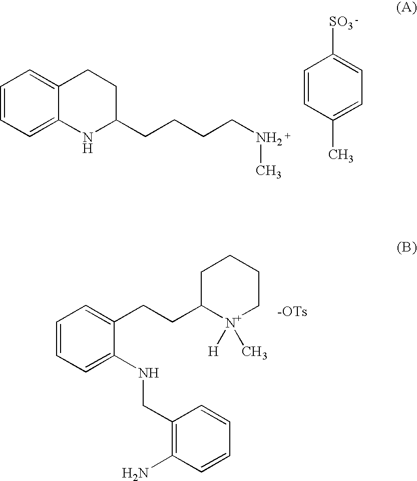

Preferably, not more than 40% of the products are by-products, for example between 30 and 40%, typically under 35%. Most preferably 30% or less are by products, e.g. 20% or less, such as 10%, for example.

The salt I is preferably in the form of substantially 1:1 stoichiometry.

### Further Steps

The product may undergo further reaction steps to functionalise or protect the aniline group (Ar—NH₂), as required, i.e. to form any of the salts containing the group NR^{a}R^{b} as disclosed herein. Subsequent reaction steps involving compounds where R^{a} and R^{b} are not both hydrogen are illustrated in U.S. Pat. No. 3,931,195, for example. Furthermore, the products of the invention may undergo further reaction steps to form compounds of formula (Y). One particular example is the reaction of compound (IIf) with cinnamoyl chloride to form the compound of formula (Y1).

Below are illustrative examples and are not intended to be limiting.

### EXAMPLES

The present invention will now be further described by way of non-limiting examples, below.

### Example 1

### Synthesis of 2-[(E)-2-(2-Nitrophenyl)ethenyl]-pyridine

A 2-L LabMax equipped with a mechanical stirrer, digital thermometer, addition funnel and a condenser with nitrogen inlet-outlet, is charged with 150.0 g of 2-nitrobenzaldehyde, 129.5 g of 2-picoline and 282 mL of acetic anhydride. Heat the mixture to an internal temperature at 138±2° C. (gentle reflux, jacket temperature 140-145° C.) over a period of 40 min and stir the reaction mixture at this temperature for an additional 28 h. Cool the dark reaction mixture to an internal temperature at 10±5° C. over a period of 1 h and add 750 mL of water over a period of at least 20 min while maintaining the internal temperature at 5-35° C. Cool the mixture to an internal temperature at 15° C. over a period of 10 min and add 262.5 g of 50% sodium hydroxide solution over a period of 40 min while maintaining the internal temperature at 15-35° C. (jacket temperature: 5° C.). Seed the reaction mixture with 120 mg of 2-[(E)-2-(2-nitrophenyl)ethenyl]-pyridine. Add 262.5 g of 50% of sodium hydroxide solution over a period of 20 min while maintaining the internal temperature at 15-35° C. Stir the reaction mixture at this temperature for an additional 1 h. Collect the solid by filtration over a Büchner funnel with suction, wash the solid with 4×375 mL of water. Dry the solid under reduced pressure (15-40 mbar) at 70° C. for 16 h until an LOD of <1% is reached to afford 195.0 g of 2-[(E)-2-(2-nitrophenyl)ethenyl]-pyridine.

Theoretical Yield: 224.6 g; % Yield: 87%; Purity: 99.5%; Melting point: 95-96° C.

### Example 2

### Synthesis of 1-Methyl-2-[(E)-2-(2-nitrophenyl)-ethenyl]-pyridinium 4-methylbenzenesulfonate

A 1-L LabMax equipped with a mechanical stirrer, digital thermometer, addition funnel and a condenser with nitrogen inlet-outlet, is charged with 80.0 g of 2-[(E)-2-(2-nitrophenyl)ethenyl]-pyridine, 350 mL of acetonitrile and 98.8 g of methyl p-toluenesulfonate. Rinse the addition funnel with 50 mL of acetonitrile and add to the reaction mixture. Heat the mixture to an internal temperature at 83±3° C. (gentle reflux, jacket temperature: 90-95° C.) over a period of 40 min and stir the reaction mixture at this temperature for an additional 24 h. Cool the mixture to an internal temperature at 35-45° C. (jacket temperature: 35-45° C.) over a period of 30 min. Heat the mixture to an internal temperature at 80±3° C. (jacket temperature: 80-83° C.) over a period of 20 min and add 400 mL of isopropyl acetate over a period of 30 min while maintaining the internal temperature at 70-83° C. (jacket temperature: 80-83° C.). Cool the suspension to an internal temperature at 20±5° C. over a period of 1 h and stir the suspension at this temperature for an additional 4 h. Collect the solid by filtration over a Büchner funnel with suction, wash the solid with 2×160 mL of isopropyl acetate. Dry the solid under reduced pressure (15-40 mbar) at 60° C. until an LOD of <1% is reached to afford 135.0 g of 1-methyl-2-[(E)-2-(2-nitrophenyl)-ethenyl]-pyridinium 4-methylbenzenesulfonate.

Theoretical Yield: 145.9 g; Yield: 92.5%; Purity: 99.7 ; Melting Point: 172-173° C.

### Example 3

### Synthesis of 2-[2-(1-Methyl-2-piperidinyl)ethyl]-benzenamine 4-methylbenzenesulfonate

Inert the MP-10 vessel by pressurizing with nitrogen to 4.5 bar¹, then depressurizing to 1 bar. Repeat this pressurization/depressurization four times. Charge the MP-10 vessel with 43.90 g of 1-methyl-2-[(E)-2-(2-nitrophenyl)-ethenyl]-pyridinium 4-ethylbenzenesulfonate. Inert the vessel with nitrogen as described above. Add 1.87 g of 10% Pt/C (62.4% wet). Inert the vessel with nitrogen as described above. Add 395.6 g of methanol. Inert the vessel with nitrogen as described above. Stir the vessel at 450 rpm, set the batch temperature at 30° C., and allow the batch temperature to equilibrate at 30° C. Set the temperature control of the RC1 to Tj mode and turn off the agitator. Purge the headspace of N₂, and replace with H₂ by pressurizing with H₂ to 4.5 bar, depressurizing to 1 bar. Repeat the H₂ pressurization/depressurization cycle 4 times. After the final depressurization, set the reactor pressure to 5.2 bar, agitate at 450 rpm to start the reaction, and switch the RC1 to Tr mode. The initial reaction is exothermic, giving a maximum heat evolution rate of about 35 W/kg (excepting for a short-lived spike with a maximum of ˜50 W/kg). Reaction start is detected immediately, based on hydrogen uptake and heat evolution. Hydrogenate at 30° C. and 5.2 bar for 7.2 h. Depressurize the reactor to 1 bar, and purge with N₂ by pressurizing to 4.5 bar and depressurizing as described above (5 cycles). Empty the reactor, and rinse the MP-10 vessel with: 44.8 g of methanol and combine the rinse with the reaction mixture. Filter the batch over a pad of 8.0 g of Celite. Wash the Celite pad with 39.6 of methanol and combine the filtrate [caution: do not allow the cake to dry; solid catalyst is flammable]. Charge the filtrate into a 1-L LabMax, distill the filtrate at an internal temperature at 35-45° C. (Tj mode, jacket temperature: 65-75° C.) under reduced pressure (80-160 mbar) to collect 450 mL of solvent (batch volume: ˜150 mL). Add to the batch 353.3 g of peroxide-free 2-propanol. Distill the batch at an internal temperature at 35-45° C. (Tj mode, jacket temperature: 65-75° C.) under reduced pressure (80-160 mbar) to collect 450 mL of solvent (batch volume: ˜150 mL). Add 353.3 g of 2-propanol. Distill the batch at an internal temperature at 35-45° C. (Tj mode, jacket temperature: 65-75° C.) under reduced pressure (80-160 mbar) to collect 450 mL of solvent (batch volume: ˜150 mL). Heat the batch to an internal temperature at 60±5° C. over a period of 20 min and add 43.7 g of isopropyl acetate over a period of 20 min while maintaining the internal temperature at 55-65° C. Cool the mixture to an internal temperature at 40±5° C. over a period of 20 min and seed the batch with 160 mg of pure A6. Cool the suspension to an internal temperature at 20±5° C. over a period of 1 h and stir at this temperature for an additional 4 h. Collect the solid by filtration over a Büchner funnel with suction, wash the solid with 2×42.1 g of 2-propanol/isopropyl acetate (1:2 v/v). Dry the solid under reduced pressure (15-40 mbar) at 60° C. until an LOD of <1% is reached to afford 26.3 g of 2-[2-(1-methyl-2-piperidinyl)ethyl]benzenamine 4-methylbenzenesulfonate (1:1).

Theoretical Yield: 41.60 g; Yield: 63.2%; Purity: 98.8%; Melting Point: 133-135° C.; MS: [MH]+219.1

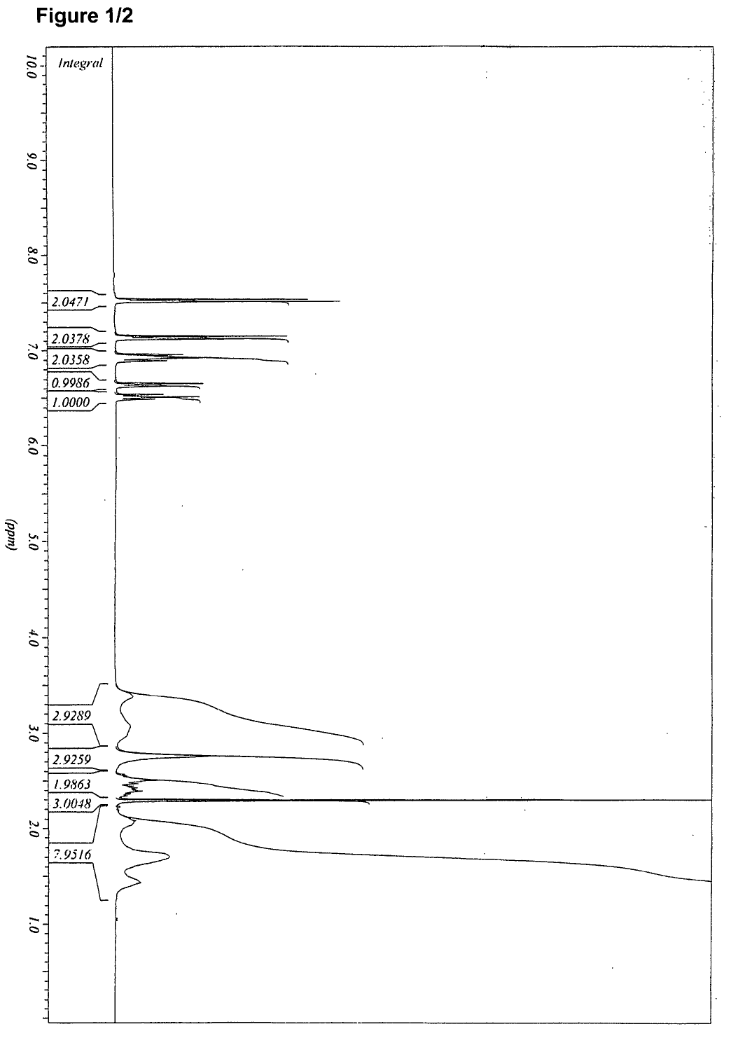

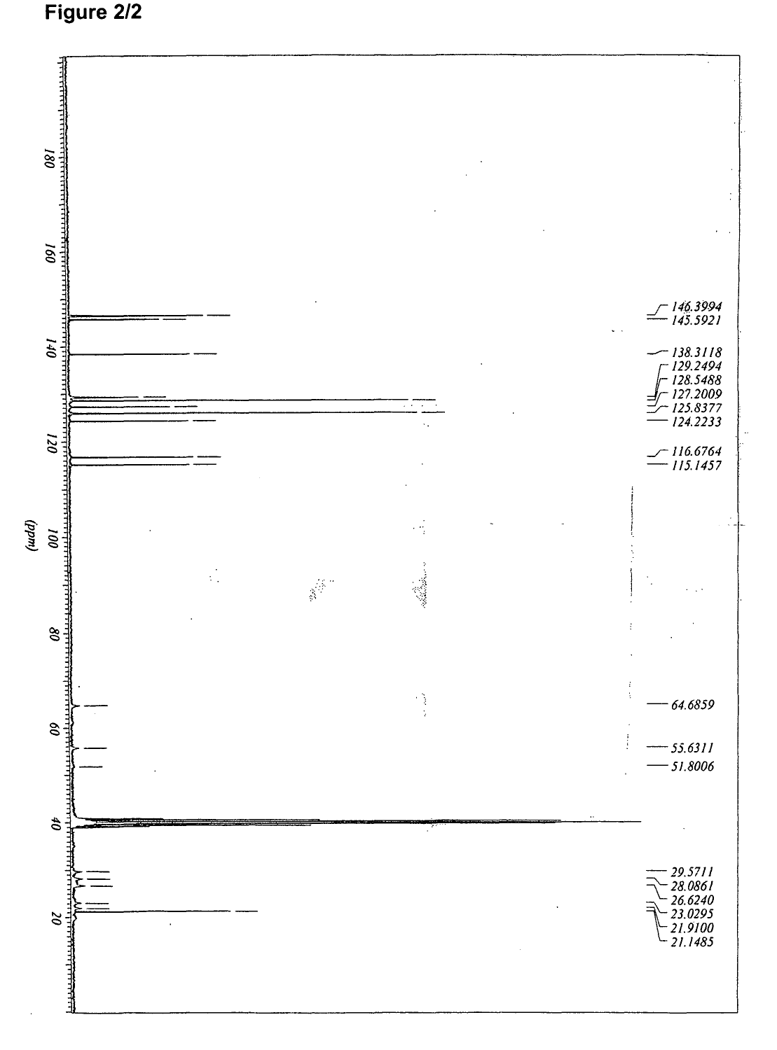

¹HNMR: (DMSO, 300 MHz): δ 7.53 (d, 2H, J=8.1 Hz), 7.13 (d, 2H, J=8.1 Hz), 6.95-6.88 (m, 2H), 6.64 (dd, 1H, J=1.0 Hz), 6.51 (dt, 1H, J=7.4 & 1.1 Hz), 3.38 (m, 1H), 3.07 (m, 2H0, 2.76 (s, 3H), 2.58-2.34 (m, 2H), 2.29 (s, 3H), 2.15-1.43 (m, 8H) (see FIG. 1). ¹³CNMR: (DMSO, 75 MHz): δ 146.4, 145.6, 138.3, 129.2, 128.6, 127.2, 125.8, 124.2, 116.7, 115.2, 64.7, 55.6, 51.8, 29.6, 28.1, 26.6, 23.0, 21.9, 21.2 (see FIG. 2).

### Example 4

### Scale Up Synthesis of 2-[(E)-2-(2-Nitrophenyl)ethenyl]-pyridine

The process was carried out, as described in Example 1, but on a larger scale using 40 kg of A1. The process afforded a yield of 47.3 kg (79%).

### Example 5

### Scale Up of Synthesis of 1-Methyl-2-[(E)-2-(2-nitrophenyl)-ethenyl]-pyridinium 4-methylbenzenesulfonate

The process was carried out as described in Example 2, but on a larger scale using 47.2 kg of A4. The process afforded 80.2 kg (93% yield) with a purity of 99%.

### Example 6

### Scale Up Synthesis of 2-[2-(1-Methyl-2-piperidinyl)ethyl]-benzenamine 4-methylbenzenesulfonate

The process was carried out as described in Example 3, but on a larger scale using 3 batches of 20 kg of A5. The process afforded a total of 40 kg (70% yield) with a purity of 99%.

1. A salt of Formula (I): 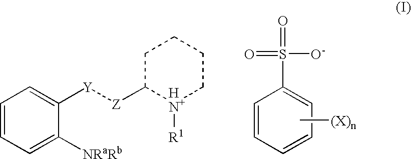
where X is an organic or inorganic moiety,
n is 0, 1, 2, 3 or 4; and
R^{a} and R^{b} are each independently selected from H, OH, C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl, or are both oxygen to produce the moiety NO₂; and
R¹ is C₁, C₂, C₃ or C₄ alkyl; and
Y and Z are both carbon; and
the broken lines [symbol] represent saturated or unsaturated bonds.

2. A salt of claim 1, wherein salt is of formula IA:

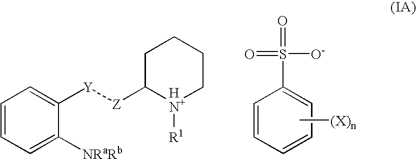

3. A salt of claim 1, wherein the salt is of formula IB:

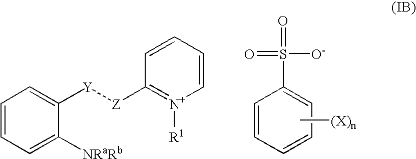

4. A salt of claim 1, wherein n is 1.

5. A salt of claim 1, which comprises an X group in the para position.

6. A salt of claim 1, wherein X is C₁, C₂, C₃ or C₄ alkyl.

7. A salt of claim 1, wherein X is methyl.

8. A salt of claim 1 where R¹ is methyl.

9. A salt of claim 1 having the formula (IIf):

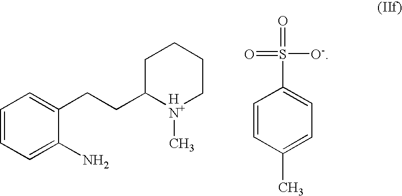

10. A salt of claim 1 having the formula (IIIf):

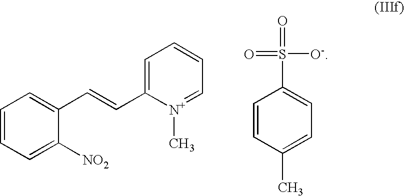

11. A salt of claim 1 as hereinbefore described in the Examples.

12. A product, for example a solution, comprising a source of cations of formula (vi) and a source of anions of formula (vii): 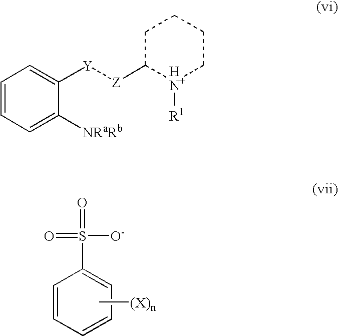
where X is an organic or inorganic moiety,
n is 0, 1, 2, 3 or 4; and
R^{a} and R^{b} are each independently selected from H, OH, C₁, C₂, C₃ or C₄ alkyl, C₁, C₂, C₃ or C₄ haloalkyl, C₁, C₂, C₃ or C₄ alkoxy, C₁, C₂, C₃ or C₄ alkenyl, or are both oxygen to produce the moiety NO₂; and
R¹ is C₁, C₂, C₃ or C₄ alkyl; and
Y and Z are both carbon; and
the broken lines [symbol] represent saturated or unsaturated bonds.

13-16. (canceled)

17. A process for manufacturing a salt of claim 1, the process comprising:
(a) reacting 2-nitrobenzaldehyde with 2-picoline to form a compound of formula (i); 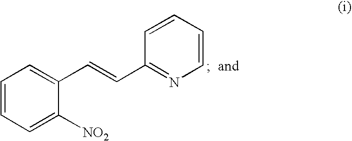
(b) converting the compound of formula (i) into a salt of any one of claims 1 to 12.

18. The process of claim 17 comprising heating the 2-nitrobenzaldehyde with 2-picoline in the presence of a dehydrating agent.

19. The process of claim 17, further comprising treating the salt of formula (i) with a base to raise the pH to 9 or more prior to converting it to said salt.

20. The process of claim 19, wherein the pH is raised to between 9 and 11.

21. The process of claim 17 wherein the salt of formula (i) is isolated.

22. The process of claim 17 wherein the salt of formula (i) is not isolated.

23. The process of claim 17, wherein the conversion the compound of formula (i) into said salt comprises treating the compound with an aryl sulphonate, where the aryl sulphonate has the formula (iv): 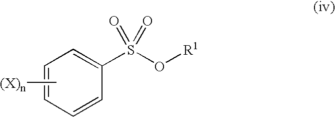
where R¹ is C₁, C₂, C₃ or C₄ alkyl; and
X is an organic or inorganic moiety; and
n is an integer of 1 to 4.

24. The process of claim 23, wherein n is 1.

25. The process of claim 23, which comprises an X group in the para position.

26. The process of claim 23, wherein X is C₁, C₂, C₃ or C₄ alkyl.

27. The process of claim 23, wherein X is methyl.

28. The process of claim 23, where R¹ is methyl.

29. The process of claim 23, wherein the alkylating agent is methyl p-toluenesulphonate.

30. The process of claim 23, comprising heating the 2-nitrobenzaldehyde with 2-picoline in a first solvent and inducing precipitation by introducing a second solvent.

31. The process of claim 30, wherein the first solvent is selected from acetone and acetonitrile.

32. The process of claim 30, wherein the second solvent is isopropylacetate.

33. A process for converting a first salt of claim 1 in which at least one broken line [symbol] represents an unsaturated bond into a second salt of any one of claims 1 to 12 in which all the broken lines represent saturated bonds, the process comprising exposing the unsaturated salt to hydrogen in the presence of a hydrogenation catalyst, for example, Pt or Pd under conditions comprising a pressure of over 5 bar and a temperature not above 40° C.

34. A process for converting a compound of formula (i) to a salt of formula (v), the process comprising treating a compound of formula (i) with a compound of formula (iv).

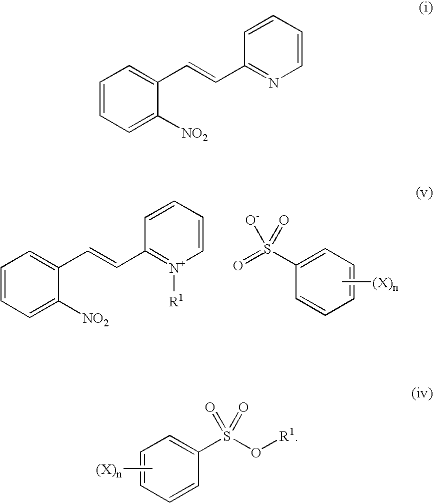

35. A process for converting a salt of formula (v) to a salt of formula (vi), the process comprising exposing a salt of formula (v) to hydrogen and a hydrogenation catalyst such as Pt or Pd:

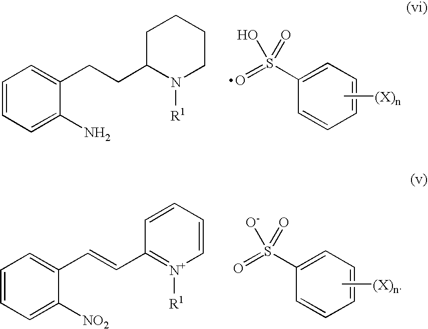

36. A process for making a 2′[2-1-(methyl-2-piperidyl) ethyl] cinnamanilide comprising further reacting a salt of formula (IIf) of claim 9 with cinnamoyl chloride.

37. The process of any of claim 36, which comprises preparing a pharmaceutical formulation from the 2′[2-1-(methyl-2-piperidyl) ethyl] cinnamanilide.

38. (canceled)

39. The process of claim 37 wherein the salt is a salt of formula (IIIe): 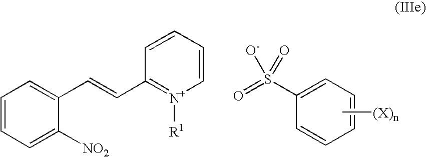
wherein X is an organic or inorganic moiety,
n is 0, 1, 2, 3 or 4; and
R¹ is C₁, C₂, C₃ or C₄ alkyl.
